(19) United States
(12) Patent Application Publication
(10) Pub. No.: US 2017/0061615 A1
(43) Pub. Date: Mar. 2, 2017

(54) IMAGE IDENTIFICATION METHOD, MAGNETIC RESONANCE IMAGING METHOD AND IMAGING APPARATUS

(71) Applicant: General Electric Company, Schenectady, NY (US)

(72) Inventors: Jia Guo, Beijing (CN); Yongchuan Lai, Beijing (CN); Xiaocheng Wei, Beijing (CN); Yi Hu, Beijing (CN)

(21) Appl. No.: 15/252,721

(22) Filed: Aug. 31, 2016

(30) Foreign Application Priority Data
Aug. 31, 2015 (CN) 201510546371.4

(51) Int. Cl. G06T 7/00; A61B 5/00; A61B 5/055

(52) U.S. Cl. CPC G06T 7/0012; G06T 7/0042; A61B 5/055; A61B 5/447; A61B 5/4561; G06T 2207/10088; G06T 2207/30196; A61B 2576/00

(57) ABSTRACT

The present invention provides an image identification method, a magnetic resonance imaging method and an imaging apparatus. Said imaging apparatus comprises: a scanning device configured to scan a target object to obtain projection images of the target object along different directions; a processing unit configured to process the obtained projection images to obtain posture feature information of the target object; a posture determining unit configured to determine a posture of the target object according to the obtained posture feature information. Therefore, the posture of the target object may be automatically recognized during the imaging, and thus the imaging operation is simplified, improving efficiency and reliability of the imaging operation.

### CROSS-REFERENCE TO RELATED APPLICATIONS

This application claims priority to Chinese patent application number 201510546371.4, filed on Aug. 31, 2015, the entirety of which is incorporated herein by reference.

### BACKGROUND

The present invention relates to an image identification method, a magnetic resonance imaging method and an imaging apparatus.

An imaging apparatus such as a computed tomography (CT) apparatus, a magnetic resonance imaging (MRI) apparatus, etc., obtains a scanning image of a target object such as a patient to be diagnosed by scanning the target object. During the scan on the target object, the target object may be made be in different postures according to the requirements. For example, a head of the patient may first enter a scanning position while the patient is supine, the head of the patient may first enter the scanning position while the patient is prostrate, or a foot of the patient may first enter the scanning position while the patient is lateral decubital. Usually, an operator such as a doctor needs to manually input information related to a posture of the target object into the imaging apparatus, and store it together with the obtained scanning image of the target object. Therefore, the operation of manually inputting the information related to the posture makes the whole imaging operation complicated. If the information input manually has an error, the target object might need to be scanned again.

### SUMMARY

An objective of exemplary embodiments of the present invention is to overcome the aforementioned and/or other problems in the prior art. Therefore, exemplary embodiments of the present invention provide an image identification method, a magnetic resonance imaging method and an imaging apparatus which may automatically identify a posture of a target object.

According to an exemplary embodiment, an image identification method may comprise: scanning a target object to obtain projection images of the target object along different directions; processing the obtained projection images to obtain posture feature information of the target object; determining a posture of the target object according to the obtained posture feature information.

According to another exemplary embodiment, a magnetic resonance imaging method may comprise: performing a magnetic resonance scan on a target object to obtain a magnetic resonance image of the target object, wherein the magnetic resonance image includes projection images of the target object along different directions; processing the obtained projection images to obtain posture feature information of the target object; determining a posture of the target object according to the obtained posture feature information.

According to yet another exemplary embodiment, an imaging apparatus may comprise: a scanning device configured to scan a target object to obtain projection images of the target object along different directions; a processing unit configured to process the obtained projection images to obtain posture feature information of the target object; a posture determining unit configured to determine a posture of the target object according to the obtained posture feature information.

Other features and aspects will be apparent through the following detailed description, figures and claims.

### BRIEF DESCRIPTION OF THE DRAWINGS

The present invention can be understood better in light of the description of exemplary embodiments of the present invention with reference to the accompanying drawings, in which:

FIG. 1 is a schematic diagram illustrating an imaging apparatus according to an exemplary embodiment;

FIG. 2 to FIG. 5 are respectively coronal plane projection images and sagittal plane projection images of a target object obtained by performing magnetic resonance imaging on the target object;

FIG. 6 is a flow chart illustrating an image identification method according to an exemplary embodiment;

FIG. 7 is a flow chart illustrating a magnetic resonance imaging (MRI) method according to an exemplary embodiment.

### DETAILED DESCRIPTION

Hereafter, a detailed description will be given for preferred embodiments of the present disclosure. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for those of ordinary skilled in the art associated with the contents disclosed in the present disclosure, which should not be regarded as insufficient disclosure of the present disclosure.

Unless defined otherwise, all the technical or scientific terms used in the Claims and the Description should have the same meanings as commonly understood by one of ordinary skilled in the art to which the present disclosure belongs. The terms “first”, “second” and the like in the Description and the Claims of the present utility model do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

In the following, exemplary embodiments will be described in details with reference to the drawings.

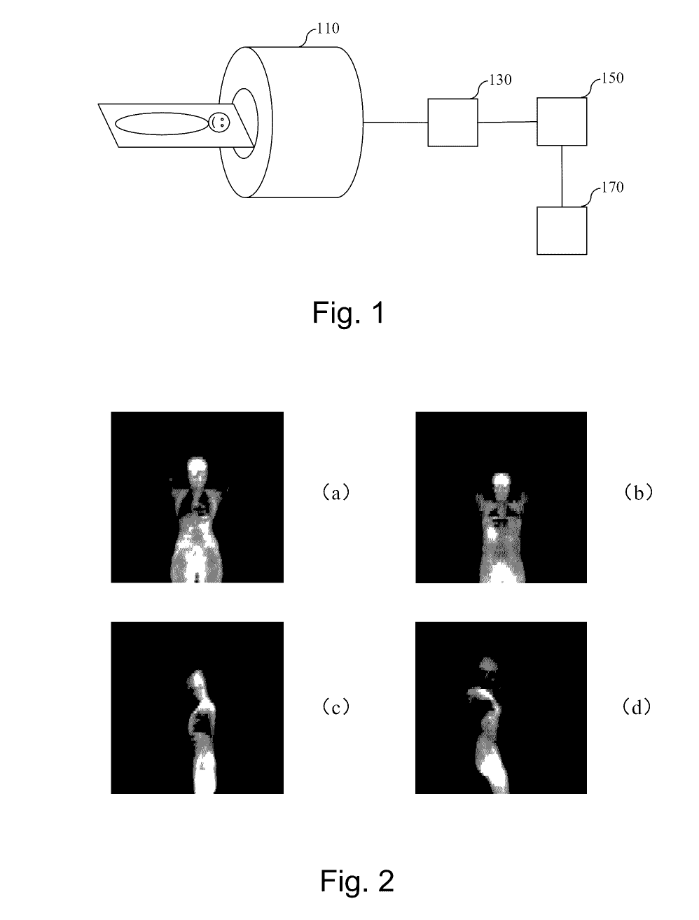

FIG. 1 is a schematic diagram illustrating an imaging apparatus according to an exemplary embodiment.

As shown in FIG. 1, the imaging apparatus according to the exemplary embodiment may include a scanning device 110, a processing unit 130 and a posture determining unit 150.

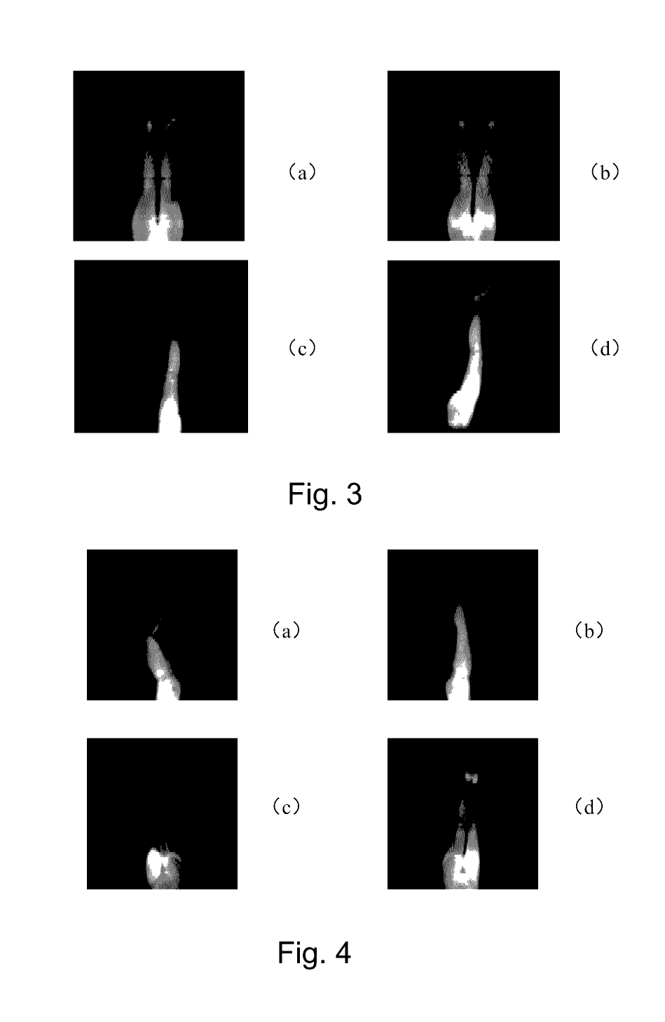

The scanning device 110 may scan a target object such as a patient (a human body) to be diagnosed to obtain an image of the target object. For example, the scanning device 110 may obtain projection images of the target object along different directions (with reference to FIG. 2 to FIG. 5). According to the present embodiment, the imaging apparatus may be implemented as a magnetic resonance imaging (MRI) apparatus. Accordingly, the scanning device 110 may be implemented as a magnetic resonance scanning device. For example, the scanning device 110 may include a main magnet for generating a static magnetic field (B0 field), a gradient coil for generating a gradient field, a radio frequency coil for generating a radio frequency field, and a receiving coil (not shown) for receiving a magnetic resonance signal from the target object to be imaged. The main magnet may include a hole, and the target object may be moved to a scanning position in the hole while being supported by a bracket such as a bed.

Such scanning device may perform a magnetic resonance imaging scan on the target object during a process that the bracket supporting the target object moves into the scanning position of the scanning device, so as to obtain the projection image of the target object.

However, the exemplary embodiment is not limited thereto, those skilled in the art may implement the imaging apparatus as multiple kinds of imaging apparatuses such as a computed tomography (CT) and the like that may obtain projection images of the target object along different directions, and thus modify the corresponding configurations of individual components thereof.

The scanning device 110 may send the obtained projection images to the processing unit 130. When the projection images are received, the processing unit 130 may process the received projection images to obtain posture feature information of the target object. Specifically, the processing unit 130 may obtain first posture feature information related to a direction of the target object and second posture feature information related to a position of the target object according to a coronal plane projection image and a sagittal plane projection image. Herein, when the target object is a human body, the first posture feature information related to the direction of the target object may include information indicating a head-foot direction of the body, and the second posture feature information related to the position of the target object includes information indicating decubitus of the body. Herein, the information indicating the head-foot direction of the body may indicate whether the head of the body first enters the scanning position or the foot of the body first enters the scanning position. This will be described in more details hereinafter with reference to FIG. 2 to FIG. 5.

The processing unit 130 may send the obtained posture feature information including the first posture feature information and the second posture feature information to the posture determining unit 150. When the posture feature information is received, the posture determining unit 150 may determine a posture of the target object according to the received posture feature information. Specifically, the posture determining unit 150 may determine the posture of the body as one of the following postures: head-foot/supination, head-foot/prostration, foot-head/supination, foot-head/prostration, head-foot/left-lying decubitus, head-foot/right-lying decubitus, foot-head/left-lying decubitus, foot-head/right-lying decubitus, according to the first posture feature information and the second posture feature information. This will be described in more details hereinafter with reference to FIG. 2 to FIG. 5.

FIG. 2 to FIG. 5 are respectively coronal plane projection images and sagittal plane projection images of a target object obtained by performing magnetic resonance imaging on the target object.

(a) and (c) in FIG. 2 are respectively a coronal plane projection image and a sagittal plane projection image obtained by the scanning device, corresponding to the target object in a first posture; (b) and (d) in FIG. 2 are respectively a coronal plane projection image and a sagittal plane projection image obtained by the scanning device, corresponding to the target object in a second posture. In the present exemplary embodiment, as an example, the scanning device 110 may have a field of view (FOV) of about 120 cm.

The processing unit 130 may obtain that the first posture feature information related to the direction of the target object is “head-foot” according to (a) in FIG. 2, i.e., the head of the target object first enters the scanning position. The processing unit 130 may obtain that the second posture feature information related to the position of the target object is “supination” according to (c) in FIG. 2, i.e., the target object is supine on the bracket. Thereby, the processing unit 130 may send the obtained first posture feature information “head-foot” and second posture feature information “supination” to the posture determining unit 150. Accordingly, the posture determining unit 150 may determine the first posture of the target object as “head-foot/supination” according to the received posture feature information.

The processing unit 130 may obtain that the first posture feature information related to the direction of the target object is “head-foot” according to (b) in FIG. 2, i.e., the head of the target object first enters the scanning position. The processing unit 130 may obtain that the second posture feature information related to the position of the target object is “prostration” according to (d) in FIG. 2, i.e., the target object is prostrate on the bracket. Thereby, the processing unit 130 may send the obtained first posture feature information “head-foot” and second posture feature information “prostration” to the posture determining unit 150. Accordingly, the posture determining unit 150 may determine the second posture of the target object as “head-foot/prostration” according to the received posture feature information.

(a) and (c) in FIG. 3 are respectively a coronal plane projection image and a sagittal plane projection image obtained by the scanning device, corresponding to the target object in a third posture; (b) and (d) in FIG. 3 are respectively a coronal plane projection image and a sagittal plane projection image obtained by the scanning device, corresponding to the target object in a fourth posture.

The processing unit 130 may obtain that the first posture feature information related to the direction of the target object is “foot-head” according to (a) in FIG. 3, i.e., the foot of the target object first enters the scanning position. The processing unit 130 may obtain that the second posture feature information related to the position of the target object is “supination” according to (c) in FIG. 3, i.e., the target object is supine on the bracket. Thereby, the processing unit 130 may send the obtained first posture feature information “foot-head” and second posture feature information “supination” to the posture determining unit 150. Accordingly, the posture determining unit 150 may determine the third posture of the target object as “foot-head/supination” according to the received posture feature information.

The processing unit 130 may obtain that the first posture feature information related to the direction of the target object is “foot-head” according to (b) in FIG. 3, i.e., the foot of the target object first enters the scanning position. The processing unit 130 may obtain that the second posture feature information related to the position of the target object is “prostration” according to (d) in FIG. 3, i.e., the target object is prostrate on the bracket. Thereby, the processing unit 130 may send the obtained first posture feature information “foot-head” and second posture feature information “prostration” to the posture determining unit 150. Accordingly, the posture determining unit 150 may determine the fourth posture of the target object as “foot-head/prostration” according to the received posture feature information.

(a) and (c) in FIG. 4 are respectively a coronal plane projection image and a sagittal plane projection image obtained by the scanning device, corresponding to the target object in a fifth posture; (b) and (d) in FIG. 4 are respectively a coronal plane projection image and a sagittal plane projection image obtained by the scanning device, corresponding to the target object in a sixth posture.

The processing unit 130 may obtain that the first posture feature information related to the direction of the target object is “foot-head” according to (c) in FIG. 4, i.e., the foot of the target object first enters the scanning position. The processing unit 130 may obtain that the second posture feature information related to the position of the target object is “left-lying decubitus” according to (a) in FIG. 4, i.e., the target object is left-lying decubital on the bracket. Thereby, the processing unit 130 may send the obtained first posture feature information “foot-head” and second posture feature information “left-lying decubitus” to the posture determining unit 150. Accordingly, the posture determining unit 150 may determine the fifth posture of the target object as “foot-head/left-lying decubitus” according to the received posture feature information.

The processing unit 130 may obtain that the first posture feature information related to the direction of the target object is “foot-head” according to (d) in FIG. 4, i.e., the foot of the target object first enters the scanning position. The processing unit 130 may obtain that the second posture feature information related to the position of the target object is “right-lying decubitus” according to (b) in FIG. 4, i.e., the target object is right-lying decubital on the bracket. Thereby, the processing unit 130 may send the obtained first posture feature information “foot-head” and second posture feature information “right-lying decubitus” to the posture determining unit 150. Accordingly, the posture determining unit 150 may determine the sixth posture of the target object as “foot-head/right-lying decubitus” according to the received posture feature information.

(a) and (c) in FIG. 5 are respectively a coronal plane projection image and a sagittal plane projection image obtained by the scanning device, corresponding to the target object in a seventh posture; (b) and (d) in FIG. 5 are respectively a coronal plane projection image and a sagittal plane projection image obtained by the scanning device, corresponding to the target object in an eighth posture.

The processing unit 130 may obtain that the first posture feature information related to the direction of the target object is “head-foot” according to (c) in FIG. 5, i.e., the head of the target object first enters the scanning position. The processing unit 130 may obtain that the second posture feature information related to the position of the target object is “left-lying decubitus” according to (a) in FIG. 5, i.e., the target object is left-lying decubital on the bracket. Thereby, the processing unit 130 may send the obtained first posture feature information “head-foot” and second posture feature information “left-lying decubitus” to the posture determining unit 150. Accordingly, the posture determining unit 150 may determine the seventh posture of the target object as “head-foot/left-lying decubitus” according to the received posture feature information.

The processing unit 130 may obtain that the first posture feature information related to the direction of the target object is “head-foot” according to (d) in FIG. 5, i.e., the head of the target object first enters the scanning position. The processing unit 130 may obtain that the second posture feature information related to the position of the target object is “right-lying decubitus” according to (b) in FIG. 5, i.e., the target object is right-lying decubital on the bracket. Thereby, the processing unit 130 may send the obtained first posture feature information “head-foot” and second posture feature information “right-lying decubitus” to the posture determining unit 150. Accordingly, the posture determining unit 150 may determine the eighth posture of the target object as “head-foot/right-lying decubitus” according to the received posture feature information.

The above has merely exemplarily described that the processing unit 130 determines the first posture feature information related to the direction of the target object according to the coronal plane projection images corresponding to the target object, but the exemplary embodiments are not limited thereto. The processing unit 130 may also determine the first posture feature information related to the direction of the target object according to the sagittal plane projection images of the target object. For example, with reference to FIG. 2, the processing unit 130 may obtain that the first posture feature information related to the direction of the target object is “head-foot” according to (c) in FIG. 2, i.e., the head of the target object first enters the scanning position.

Returning to refer to FIG. 1, although the processing unit 130 and the posture determining unit 150 are shown as two separate hardware devices in FIG. 1, the exemplary embodiment is not limited thereto. For example, the posture determining unit 150 may be integrated in the processing unit 130 as a part of the processing unit 130, or may be software or computer readable codes for achieving the above functions.

Moreover, as shown in FIG. 1, the imaging apparatus according to the exemplary embodiment may further include a memory 170. The information related to the posture of the target object determined by the posture determining unit may be stored in the memory 170 together with the obtained magnetic resonance image.

Therefore, according to the exemplary embodiment, the posture of the target object may be automatically identified during the imaging, and the identified posture may be as a note added into the image of the target object or be stored in the memory together with the image of the target object. Therefore, the posture may not need to be manually input, thus an imaging operation is simplified, improving efficiency and reliability of the imaging operation. In addition, performing imaging again due to manually inputting a wrong posture may be avoided.

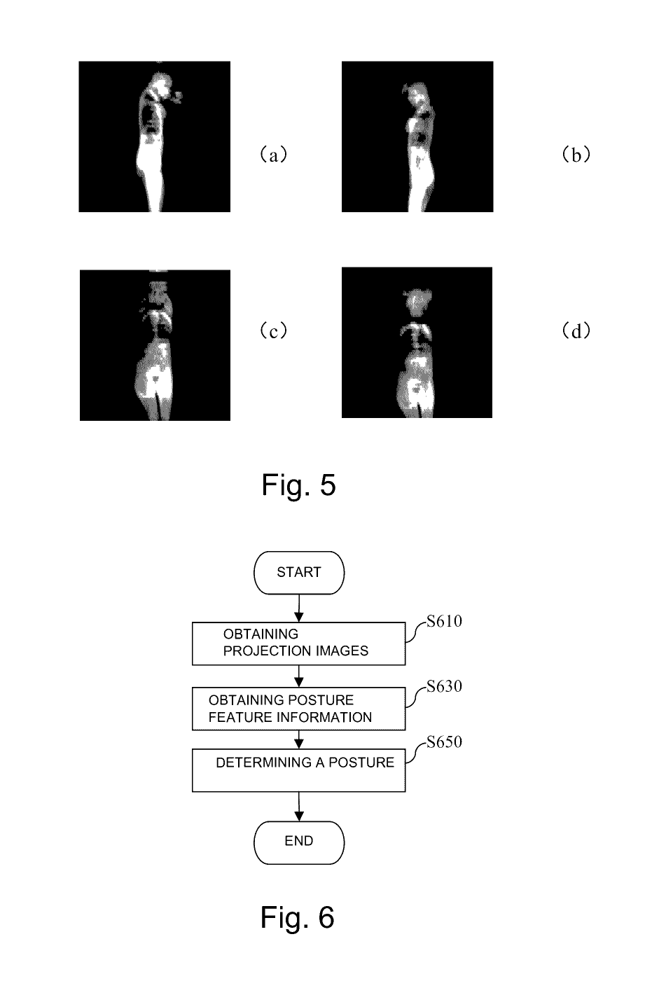

FIG. 6 is a flow chart illustrating an image identification method according to an exemplary embodiment.

As shown in FIG. 6, in Step S610, a target object may be scanned to obtain projection images of the target object along different directions. Herein, the projection images may include a coronal plane projection image and a sagittal plane projection image.

Then, in Step S630, the obtained projection images may be processed to obtain posture feature information of the target object. Specifically, the coronal plane projection image and the sagittal plane projection image may be processed to obtain first posture feature information related to a direction of the target object and second posture feature information related to a position of the target object. In one exemplary embodiment, the target object may be a human body. In this way, the first posture feature information related to the direction of the target object may include information indicating a head-foot direction of the body, and the second posture feature information related to the position of the target object may include information indicating decubitus of the body.

After the posture feature information is obtained, in Step S650, a posture of the target object may be determined according to the obtained posture feature information. For example, when the target object is a human body, the posture of the body may be determined as one of the following postures: head-foot/supination, head-foot/prostration, foot-head/supination, foot-head/prostration, head-foot/left-lying decubitus, head-foot/right-lying decubitus, foot-head/left-lying decubitus, foot-head/right-lying decubitus, according to the obtained first posture feature information and second posture feature information.

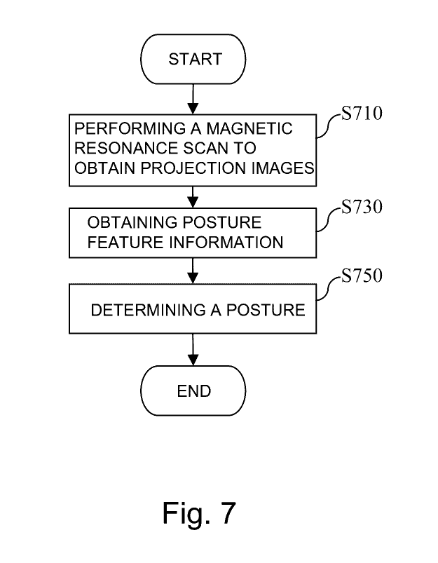

FIG. 7 is a flow chart illustrating a magnetic resonance imaging (MRI) method according to an exemplary embodiment.

As shown in FIG. 7, in Step S710, a magnetic resonance scan may be performed on a target object to obtain a magnetic resonance image of the target object. The magnetic resonance image may include projection images of the target object along different directions. For example, the projection images may include a coronal plane projection image and a sagittal plane projection image. In one exemplary embodiment, the magnetic resonance imaging scan may be performed on the target object during a process that a bracket supporting the target object moves into a scanning position of a magnetic resonance imaging apparatus, so as to obtain the projection images of the target object.

Then, in Step S730, the obtained projection images may be processed to obtain posture feature information of the target object. Specifically, the coronal plane projection image and the sagittal plane projection image may be processed to obtain first posture feature information related to a direction of the target object and second posture feature information related to a position of the target object. When the target object is a human body, the first posture feature information related to the direction of the target object may include information of a head-foot direction indicating that the head or foot of the body first enters the scanning position, and the second posture feature information related to the position of the target object may include information indicating decubitus of the body on the bracket.

After the posture feature information is obtained, in Step S750, a posture of the target object may be determined according to the obtained posture feature information. For example, when the target object is a human body, the posture of the body may be determined as one of the following postures: head-foot/supination, head-foot/prostration, foot-head/supination, foot-head/prostration, head-foot/left-lying decubitus, head-foot/right-lying decubitus, foot-head/left-lying decubitus, foot-head/right-lying decubitus, according to the obtained first posture feature information and second posture feature information.

According to the exemplary embodiment, the posture of the target object may be automatically identified during the imaging. Hence, the posture may not need to be manually input, thus an imaging operation is simplified, improving efficiency and reliability of the imaging operation. In addition, performing imaging again due to manually inputting a wrong posture may be avoided.

Some exemplary embodiments have been described in the above. However, it should be understood that various modifications may be made thereto. For example, if the described techniques are carried out in different orders, and/or if the components in the described system, architecture, apparatus or circuit are combined in different ways and/or replaced or supplemented by additional components or equivalents thereof, proper results may still be achieved. Accordingly, other implementation also falls within a protection range of the Claims.

What is claimed is:

1. An image identification method, comprising:
scanning a target object to obtain projection images of the target object along different directions;
processing the obtained projection images to obtain posture feature information of the target object; and
determining a posture of the target object according to the obtained posture feature information.

2. The method according to claim 1, wherein the projection images comprise a coronal plane projection image and a sagittal plane projection image.

3. The method according to claim 2, wherein the step of processing the projection images to obtain posture feature information comprises:
processing the coronal plane projection image and the sagittal plane projection image to obtain first posture feature information related to a direction of the target object and second posture feature information related to a position of the target object.

4. The method according to claim 3, wherein the target object is a human body, the first posture feature information related to the direction of the target object comprises information indicating a head-foot direction of the body, and the second posture feature information related to the position of the target object comprises information indicating decubitus of the body.

5. The method according to claim 4, wherein the step of determining a posture of the target object according to the posture feature information comprises:
according to the obtained first posture feature information and second posture feature information, determining the posture of the body as one of the following postures:
head-foot/supination, head-foot/prostration, foot-head/supination, foot-head/prostration, head-foot/left-lying decubitus, head-foot/right-lying decubitus, foot-head/left-lying decubitus, foot-head/right-lying decubitus.

6. A magnetic resonance imaging method, comprising:
performing a magnetic resonance scan on a target object to obtain magnetic resonance images of the target object, wherein the magnetic resonance images comprise projection images of the target image along different directions;
processing the obtained projection images to obtain posture feature information of the target object; and
determining a posture of the target object according to the obtained posture feature information.

7. The method according to claim 6, wherein the step of obtaining the magnetic resonance images comprises:
performing a magnetic resonance imaging scan on the target object during a process that a bracket supporting the target object moves into a scanning position of a magnetic resonance imaging apparatus, so as to obtain the projection images of the target object.

8. The method according to claim 7, wherein the projection images comprise a coronal plane projection image and a sagittal plane projection image.

9. The method according to claim 8, wherein the step of processing the projection images to obtain posture feature information comprises:
processing the coronal plane projection image and the sagittal plane projection image to obtain first posture feature information related to a direction of the target object and second posture feature information related to a position of the target object.

10. The method according to claim 9, wherein the target object is a human body, the first posture feature information related to the direction of the target object comprises information of head-foot direction indicating that a head or foot of the body first enters the scanning position, and the second posture feature information related to the position of the target object comprises information indicating decubitus of the body on the bracket.

11. The method according to claim 10, wherein the step of determining a posture of the target object according to the posture feature information comprises:
according to the obtained first posture feature information and second posture feature information, determining the posture of the body as one of the following postures:
head-foot/supination, head-foot/prostration, foot-head/supination, foot-head/prostration, head-foot/left-lying decubitus, head-foot/right-lying decubitus, foot-head/left-lying decubitus, foot-head/right-lying decubitus.

12. The method according to claim 6, further comprising:
storing information related to the determined posture of the target object together with the obtained magnetic resonance image.

13. An imaging apparatus, comprising:
a scanning device configured to scan a target object to obtain projection images of the target object along different directions;
a processing unit configured to process the obtained projection images to obtain posture feature information of the target object;
a posture determining unit configured to determine a posture of the target object according to the obtained posture feature information.

14. The imaging apparatus according to claim 13, wherein the projection images comprise a coronal plane projection image and a sagittal plane projection image.

15. The imaging apparatus according to claim 14, wherein the processing unit is configured to process the coronal plane projection image and the sagittal plane projection image to obtain first posture feature information related to a direction of the target object and second posture feature information related to a position of the target object.

16. The imaging apparatus according to claim 15, wherein the first posture feature information related to the direction of the target object comprises information of head-foot direction indicating that a head or foot of a human body first enters a scanning position, and the second posture feature information related to the position of the target object comprises information indicating decubitus of the body on a bracket.

17. The imaging apparatus according to claim 16, wherein the posture determining unit is configured to determine the posture of the body as one of the following postures:
head-foot/supination, head-foot/prostration, foot-head/supination, foot-head/prostration, head-foot/left-lying decubitus, head-foot/right-lying decubitus, foot-head/left-lying decubitus, foot-head/right-lying decubitus, according to the obtained first posture feature information and second posture feature information.

18. The imaging apparatus according to claim 13, wherein said imaging apparatus is a magnetic resonance imaging apparatus, and the scanning device is configured to perform a magnetic resonance imaging scan on the target object during a process that a bracket supporting the target object moves into a scanning position of the scanning device, so as to obtain a magnetic resonance image including the projection images of the target object.

19. The imaging apparatus according to claim 18, further comprising:
a memory configured to store information related to the determined posture of the target object together with the obtained magnetic resonance image.
